(19) United States
(12) Patent Application Publication
(10) Pub. No.: US 2016/0206287 A1
(43) Pub. Date: Jul. 21, 2016

(54) Wearable Doppler Ultrasound Based Cardiac Monitoring

(71) Applicant: Yoram Palti, Haifa (IL)

(72) Inventor: Yoram Palti, Haifa (IL)

(21) Appl. No.: 14/994,089

(22) Filed: Jan. 12, 2016

Related U.S. Application Data
(60) Provisional application No. 62/103,633, filed on Jan. 15, 2015.

(51) Int. Cl. A61B 8/08; A61B 8/02

(52) U.S. Cl. CPC A61B 8/488; A61B 8/02

(57) ABSTRACT

The operation of a heart of a patient is monitored by transmitting ultrasound energy into the lungs of the patient, receiving ultrasound energy reflected from the lungs of the patient, detecting Doppler shifts in the received reflections, and processing the Doppler shifts into power and velocity data. Cardiac cycles are identified based on the power and velocity data and a determination when an identified cardiac cycle is abnormal is made. When an abnormal cardiac cycle is encountered, data corresponding to the abnormal cardiac cycle is stored. The data that was stored is eventually output. Optionally, abnormal cardiac cycles are identified using match filtering.

### CROSS REFERENCE TO RELATED APPLICATIONS

This application claims the benefit of U.S. Provisional Application 62/103,633, filed Jan. 15, 2015, which is incorporated herein by reference in its entirety.

### BACKGROUND

A conventional Holter monitor is a small portable, wearable, battery operated device designed to record and store a person's ECG continuously while he maintains his normal daily routine and even during exercise. The ECG recording is usually done using 3-9 patch electrodes fixed to the chest skin by appropriate adhesive. Each electrode is connected by insulated wire leads to the monitor that includes the ECG amplifiers, data storage and analysis, etc. It may be worn around the neck or attached to a belt. Most often the recording duration is 24-48 hours. Some systems, that use large capacity memory storage, can be used for longer periods of time. The data thus collected is usually analyzed offline, but some analysis may be carried out by the device itself during use.

A Holter monitor test is usually performed after a traditional cardiac rhythm test doesn't provide enough information about the heart's condition. Holter monitors are typically used for cardiac rhythm monitoring. As such they may be used to diagnose atrial fibrillation and flutter, multifocal atrial tachycardia, Paroxysmal supraventricular tachycardia, Extra systoles, Bradycardia, etc.

While Holter monitors are in wide use, they are associated with a number of serious deficiencies, primarily relating to discomfort to the patient and technical faults. Patient discomfort is mainly due to the numerous electrode patches and to the associated wiring. In view of this fact, the monitoring duration is often too short and often a sub-optimum number of electrodes (e.g., 3 electrodes) are used, both factors leading to difficulty in detecting certain arrhythmias such as atrial fibrillation and paroxysmal events. In addition, the signature of atrial fibrillation in the ECG recordings is small relative to the noise. This makes atrial fibrillation difficult to identify, especially as the appearance of the fibrillation is often transient and rare. Additional important issues relate to bad recording quality due to bad signals and noise or artifacts. These problems mostly result from patient movement which affects the signal quality and introduces electric noise (including muscle electric activity). Furthermore, electrodes often lose good contact with skin, in which case noise becomes a very serious problem. In addition often there is interference from electrically noisy environments. Noisy records strongly affect automatic signal analysis and may also make it very difficult or even impossible to analyze manually.

When the recording of ECG signals is finished (usually after 24 or 48 hours), it is up to the physician or trained technical staff to perform the signal analysis. Since it would be extremely time demanding to browse through such a long signal, there often is an integrated automatic analysis process in each Holter software which automatically identifies different types of heart beats, rhythms, etc., creates a registry, and displays suspected segments. However the success of the automatic analysis is strongly dependent on signal quality. The quality is strongly affected by the quality of the attachment of the electrodes to the patient body. Furthermore, when the patient moves, additional distortion is introduced. Such noisy records are very difficult to process.

The automatic analysis commonly provides the physician with information about ECG morphology, heart beat morphology, beat interval measurement, heart rate variability, rhythm overview, and patient diary. Advanced systems also perform spectral analysis, ischemic burden evaluation, graphs of patient's activity, or PQ segment analysis.

Most Holter devices monitor the ECG using just two or three channels. Today's trend is to minimize the number of leads to maximize the patient's comfort during recording. Although 2-3 channel recording has been used for a long time in the Holter monitoring history, using such a small number of electrodes results in relatively low accuracy. Recently 12 lead Holter monitors have also appeared. These systems use the classic Mason-Likar lead system, thus producing the signal in the same representation as during the common rest ECG and/or stress test measurement. However, recordings from these 12-lead monitors often have significantly lower resolution than those from a standard 12-lead ECG.

Modern Holter units typically record an EDF-file onto digital flash memory devices, etc. The data is uploaded into a computer which then automatically analyzes the input, counting ECG complexes, calculating summary statistics such as average heart rate, minimum and maximum heart rate, and detecting areas in the recording worthy of further study by the technician or physician.

### SUMMARY OF THE INVENTION

One aspect of the invention is directed to an apparatus for monitoring the operation of a heart of a patient. This apparatus includes an ultrasound transducer configured to transmit ultrasound energy into the lungs of the patient and receiving ultrasound energy reflected from the lungs of the patient. It also includes an ultrasound processor configured to detect Doppler shifts in the received reflections and process the Doppler shifts into power and velocity data and a memory configured to store data. It also includes a processor configured to identify cardiac cycles based on the power and velocity data, determine when an identified cardiac cycle is abnormal, store data corresponding to the abnormal cardiac cycle in the memory when a cardiac cycle is abnormal, and output the stored data.

In some embodiments, the processing of Doppler shifts into power and velocity data is implemented using an algorithm designed to increase signal from moving borders between blood vessels in the lung and air filled alveoli that surround the blood vessels (with respect to other reflected ultrasound signals). In some embodiments the processor is further configured to identify features in a plurality of cardiac cycles, and the features in any given cardiac cycle are identified after the given cardiac cycle has been identified. In some embodiments, the processor is further configured to identify a nature of the abnormality after making the determination that a cardiac cycle is abnormal. In some embodiments, the processor is further configured to identify cardiac cycles by determining an envelope of the power and velocity data and identify cardiac cycles based on the determined envelope.

In some embodiments, the processor is further configured to determine when an identified cardiac cycle is abnormal by match filtering using a match filter kernel that corresponds to a normal heartbeat. Optionally, this match filter kernel includes a first feature that corresponds to systole, a second feature that corresponds to diastole, and a third feature that corresponds to atrial contraction.

In some embodiments, the processor is further configured to determine when an identified cardiac cycle is abnormal by match filtering using a first match filter kernel when the patient's heartrate is below a threshold rate, and match filtering using a second match filter kernel when the patient's heartrate is above the threshold rate. Optionally, the first match filter kernel includes a first feature that corresponds to systole, a second feature that corresponds to diastole, and a third feature that corresponds to atrial contraction. The second match filter kernel includes a first feature that corresponds to systole and a second feature that corresponds to diastole but does not include a feature that corresponds to atrial contraction.

In some embodiments, the processor is further configured to determine when an identified cardiac cycle is abnormal by determining when the identified cardiac cycle includes at least one of atrial fibrillation and atrial flutter.

Another aspect of the invention is directed to a method of monitoring the operation of a heart of a patient. This method includes the steps of transmitting ultrasound energy into the lungs of the patient, receiving ultrasound energy reflected from the lungs of the patient, detecting Doppler shifts in the received reflections, and processing the Doppler shifts into power and velocity data. This method also includes the steps of identifying cardiac cycles based on the power and velocity data, determining when an identified cardiac cycle is abnormal, storing, when a determination is made that a cardiac cycle is abnormal, data corresponding to the abnormal cardiac cycle, and outputting the data that was stored.

In some embodiments, the step of processing the Doppler shifts into power and velocity data includes an algorithm designed to increase signal from moving borders between blood vessels in the lung and air filled alveoli that surround the blood vessels, with respect to other reflected ultrasound signals. Some embodiments further include the step of identifying features in a plurality of cardiac cycles, and the features in any given cardiac cycle are identified after the given cardiac cycle has been identified. Some embodiments further include the step of identifying, after a determination is made that a cardiac cycle is abnormal, a nature of the abnormality. In some embodiments, the step of identifying cardiac cycles includes the steps of determining an envelope of the power and velocity data and identifying cardiac cycles based on the determined envelope.

In some embodiments, the step of determining when an identified cardiac cycle is abnormal includes the step of match filtering using a match filter kernel that corresponds to a normal heartbeat. Optionally, the match filter kernel includes a first feature that corresponds to systole, a second feature that corresponds to diastole, and a third feature that corresponds to atrial contraction. In some embodiments, the step of determining when an identified cardiac cycle is abnormal includes the steps of match filtering using a first match filter kernel when the patient's heartrate is below a threshold rate, and match filtering using a second match filter kernel when the patient's heartrate is above the threshold rate. Optionally, the first match filter kernel includes a first feature that corresponds to systole, a second feature that corresponds to diastole, and a third feature that corresponds to atrial contraction, and the second match filter kernel includes a first feature that corresponds to systole and a second feature that corresponds to diastole but does not include a feature that corresponds to atrial contraction.

In some embodiments, the step of determining when an identified cardiac cycle is abnormal includes the step of determining when the identified cardiac cycle includes at least one of atrial fibrillation and atrial flutter.

### BRIEF DESCRIPTION OF THE DRAWINGS

FIG. 1 depicts a transducer 3 that is used with the system.

FIG. 2A is a block diagram of a first embodiment of the invention.

FIG. 2B is a block diagram of a second, integrated, embodiment of the invention.

FIG. 3A depicts the power and velocity Doppler data for a normal heartbeat.

FIG. 3B depicts the power and velocity Doppler data for heartbeats with an atrial extra systole of sinus origin.

FIG. 3C depicts the power and velocity Doppler data for heartbeats with a ventricular extra systole.

FIG. 3D depicts the power and velocity Doppler data for a patient with atrial fibrillation.

FIG. 3E depicts the power and velocity Doppler data for a patient with atrial flutter.

FIG. 4 is a schematic representation of the basic data handling procedure that is implemented by the processor.

FIG. 5 is an example of LDS power and velocity data for a series of four heartbeats.

FIGS. 6A and 6B depict templates for use in some embodiments.

FIGS. 7A and 7B depict templates for use in other embodiments.

FIGS. 8A and 8B depict feature definitions for the embodiments of FIGS. 6A and 6B.

FIGS. 9A and 9B depict feature definitions for the embodiments of FIGS. 7A and 7B.

FIG. 10A depicts the identified features for a normal heartbeat.

FIG. 10B depicts the identified features for atrial extra systole arrhythmias.

FIG. 10C depicts the identified features for ventricular extra systole arrhythmias.

FIG. 10D depicts the identified features for atrial fibrillation arrhythmias.

FIG. 10E depicts the identified features for atrial flutter arrhythmias.

FIGS. 11A and 11B represent the performance measures obtained by an SVM classifier for recognizing atrial fibrillation.

FIG. 12 provides an example of how the readings obtained from both an LDS-based system and a conventional ECG-based system are affected by patient movement.

### DESCRIPTION OF THE PREFERRED EMBODIMENTS

The embodiments described below, which are referred to herein as “D-Holter” (for Doppler based Holter) minimizes most of the problems associated with standard Holter devices. D-Holter uses Doppler ultrasound sonograms (DCG for Doppler Cardiogram) instead of the electric signal registration used in conventional ECG-based Holter devices. D-Holter is based on the inventor's finding that transthoracic Doppler aimed at the lungs can detect signals that reflect cardiac activity, as described in Y. Palti et al., Pulmonary Doppler Signals: a Potentially New Diagnostic Tool, Eur J Echocardiography 12; 940-944 (2011); and Y. Palti et al., Footprints of Cardiac Mechanical Activity as Expressed in Lung Doppler Signals, Echocardiography 32(3):407-410 (2015). Doppler signals obtained from a human lung are referred to herein as Lung Doppler Signals, or LDS, and they are in synchrony with the cardiac cycle. An explanation of LDS is provided in U.S. patent application Ser. No. 12/912,988 (filed Oct. 27, 2010), which is incorporated herein by reference in its entirety. That application (which was published as US2011/0125023) describes detecting Doppler shifts of reflected ultrasound induced by moving borders between blood vessels in the lung and air filled alveoli that surround the blood vessels, and that the movement of the border is caused by pressure waves in the blood vessels that result in changes in diameter of those blood vessels. That application also describes approaches for processing the detected Doppler shifts with an algorithm designed to increase signal from the moving border with respect to other reflected ultrasound signals.

Doppler ultrasound is used to determine the power at every relevant velocity in a target region of the subject, over time. This is accomplished by generating pulsed ultrasound beams, picking up the reflected energy, calculating the Doppler shifts as well as phase shifts, and processing the data thus obtained to provide the matrix of power and corresponding velocities of the ultrasound reflectors.

The embodiments described herein are similar to conventional TCD systems in that the ultrasound beam is directly aimed at the known location of the target, without relying on imaging. The front end and data acquisition portion of the embodiments described herein are preferably configured similarly to a conventional Trans Cranial Doppler (TCD) pulsed Doppler systems. One example of such a system is the Sonara/tek pulsed Trans-Cranial-Doppler device. Note that in the Sonara/tek system, the acquired data is sent to an external computer that is loaded with software to generate a conventional Doppler ultrasound display (e.g., on a monitor associated with the computer) in which the x axis represents time, the y axis represents velocity, and power is represented by color. But the functionality of this external computer and display is not implemented in the embodiments described herein.

The embodiments described herein are also similar to TCD systems because they preferably use a relatively wide beam. For example, beams with an effective cross section of at least ½ cm are preferred (e.g., between ½ and 3 cm) may be used. This may be accomplished by using a smaller transducer, and by using single element transducers instead of phased array transducers that are popular in other anatomical applications. When a wider beam is used, the system can take advantage of the fact that the lungs contain relatively large complexes of unspecified geometrical shape consisting of blood vessels (both arteries and veins) and their surrounding lung tissues. For example, the same transducers that are used in standard TCD probes (like those available for use with the Sonara/tek machine) may be used, such as a 21 mm diameter, 2 MHz sensor with a focal length of 4 cm.

In alternative embodiments, conventional probes for making Doppler ultrasound measurements of peripheral or cardiac blood vessels may also be used. But those probes are less preferred because they typically have narrow beams, often shaped using a phased array transducer, to provide a high spatial resolution that is helpful for making geometrical characterization of the relatively small targets.

Note that since imaging the lung with ultrasound is impossible because of the scattering, one has to scan for targets without guidelines, except for the known anatomy. But this is not problematic because LDS can be obtained from any territory of the lungs, and the lungs are large and have a known location. Note also that scattering lowers the advantage of scanning by either phase array or by mechanical means. Furthermore, since the whole lung depth induces scattering, CW (continuous wave) ultrasound is less effective than PW (pulsed wave) Doppler ultrasound for pulmonary applications. Therefore, some preferred embodiments utilize PW ultrasound with relatively wide beams.

The D-Holter is preferably a battery operated wearable device that transmits ultrasound energy from a specially designed patch-mounted transducer, and registers and analyses the ultrasound energy reflected back from a human body.

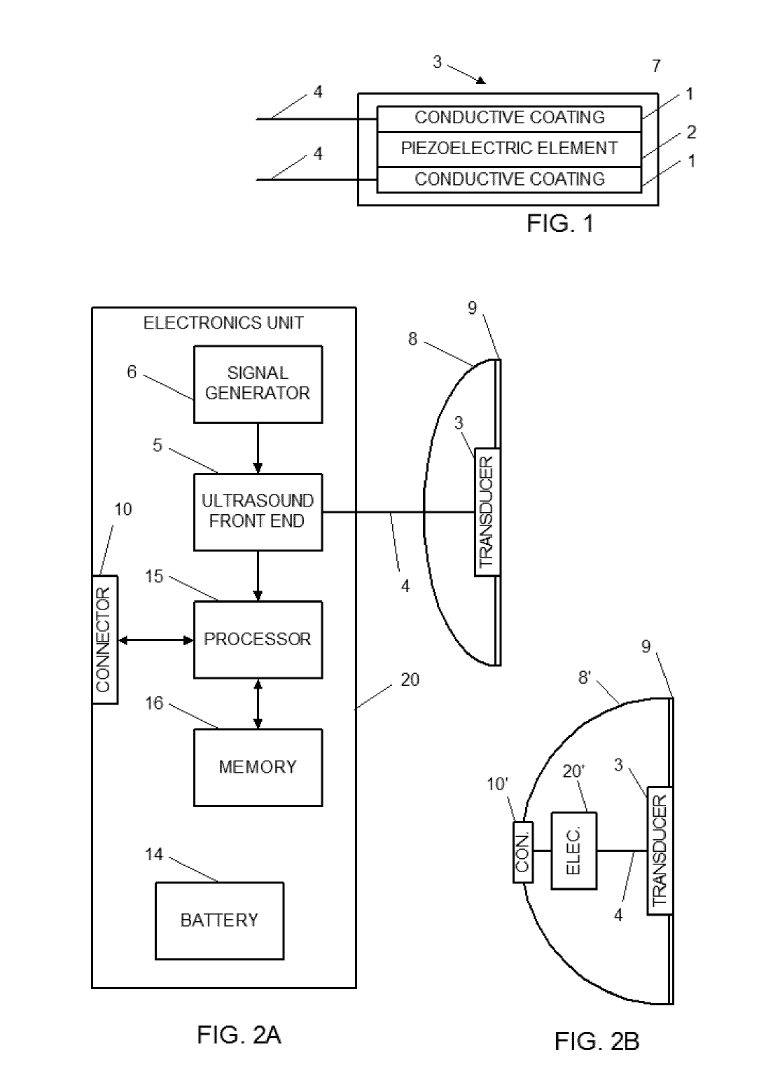

FIG. 1 depicts a transducer 3 that is an integral part of the system. The transducer 3 is preferably made from a thin flat piezoelectric element 2 (e.g., between 0.1-1 mm thick) such as a ceramic disk, with a diameter preferably in the range of 0.5-5 cm, or between 1 and 3 cm. The piezoelectric element 2 is activated by applying electrical signals to two thin conductive coatings 1 covering each of its two faces. The two conductive coatings 1 are separated by the piezoelectric element such that they are electrically isolated from each other. A relatively thin biocompatible electric insulator 7 completely covers the whole transducer 3 such that there is no current leakage to the body surface or the person handling the device. Lead wires 4 are connected to each of the conductive coatings 1 so the transducer can be driven and so that return signals from the transducer can received.

FIG. 2A depicts a first embodiment for implementing the D-Holter using a two-part system. The first part is the electronics unit 20, and the second part is the transducer 3 described above in connection with FIG. 1. The transducer 3 is preferably encapsulated within a biocompatible casing 8 that is fixed to the chest wall using an appropriate adhesive 9 similar to the adhesives used for ECG electrode. Taken together, the transducer 3 in the casing 8 resembles a patch. The transducer 3 is connected to the electronics unit via a cable that contains the leads 4. The electronics unit is preferably worn by the patient, e.g., by hooking it on to the patient's belt or by hanging it like a pendant around the patient's neck.

The electronics unit 20 includes a signal generator 6 that generates appropriate signals for driving the ultrasound transducer. Suitable signals include pulsed AC signals ranging from 1-4 MHz. In some preferred embodiments, pulsed AC signals with a frequency of about 2 MHz is used. The signal from the signal generator 6 is amplified and sent to the transducer 3 via the ultrasound front end 5, and the amplified signal is delivered to the transducer 3 via the leads 4, to excite the transducer. A suitable pulse duration for use this embodiment will typically be 2-10 microseconds (more preferably 2-5 μSec), with a repetition rate 100-3000 Hz, (more preferably 100-1000 Hz). This repetition rate is sufficiently high to be consistent with the Nyquist criterion rate for measuring Doppler shifts corresponding to velocities of 10-15 cm/sec.

The ultrasound waves reflected back from body reflectors that are moving relative to the transducer 3 are picked up by the transducer 3. They are amplified and digitized in the ultrasound front end 5 and converted into power and velocity data in a conventional manner. The power and velocity data is delivered to the processor 15, which is programmed to implement the algorithms described below. The processor has access to memory 16 for storing any data that will ultimately be delivered to the health care provider. The data stored in memory 16 can be delivered via a wired connection via connector 10, and/or via a wireless connection (e.g., Bluetooth). A battery 14 provides power for the entire device.

Optionally, battery power can be conserved by using shorter pulse durations and lower repetition rates (within the confines of the Nyquist criterion discussed above). Rechargeable or interchangeable batteries may be used to reduce the size and weight of the electronics unit 20 (as compared, for example, including a battery designed to last for a full two weeks).

The FIG. 2B embodiment is another preferred embodiment in which the transducer 3 and all of the components that were located in the electronics unit 20 of the FIG. 2A embodiment (including the battery 14) are housed in the electronics unit 20′ located within a larger patch-shaped housing 8′ in order to provide a stand-alone system. In a variation of the FIG. 2B embodiment (not shown), the patch-shaped housing includes all of the components that are located in the patch-shaped housing 8′ of the FIG. 2B embodiment except for the battery 14. In this variation, the battery is housed externally to the patch shaped housing and is connected via a cable.

Advantageously, in both the FIG. 2A and FIG. 2B embodiments, only one adhesive connection point to the patient's body is required. This stands in contrast conventional ECG-based Holter systems, which typically use between 3 and 12 adhesive connection points to the patient's body, and require a larger number of connection points in order to achieve improved accuracy. When a large number of electrodes is used, the electrode array will be uncomfortable for long term monitoring, and may interfere with the patient's ability to sleep. In contrast, an LDS-based system requires only a single adhesive connection to the patient. This less intrusive approach provides improved comfort for long term monitoring, which is particularly important for those situations that require continuous monitoring over the course of one or more weeks (e.g., diagnosing atrial fibrillation and atrial flutter).

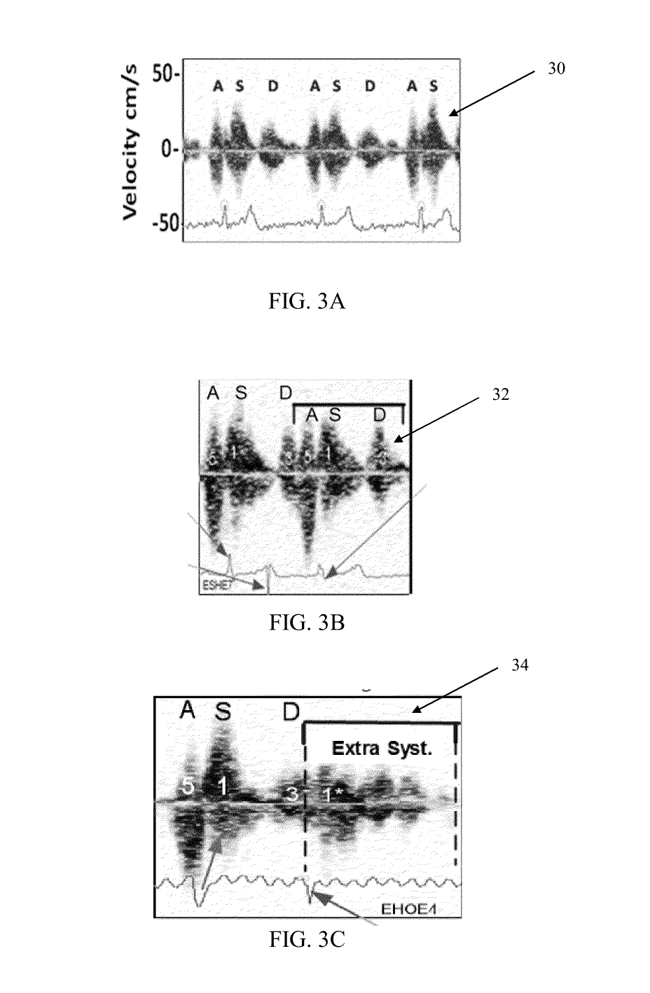

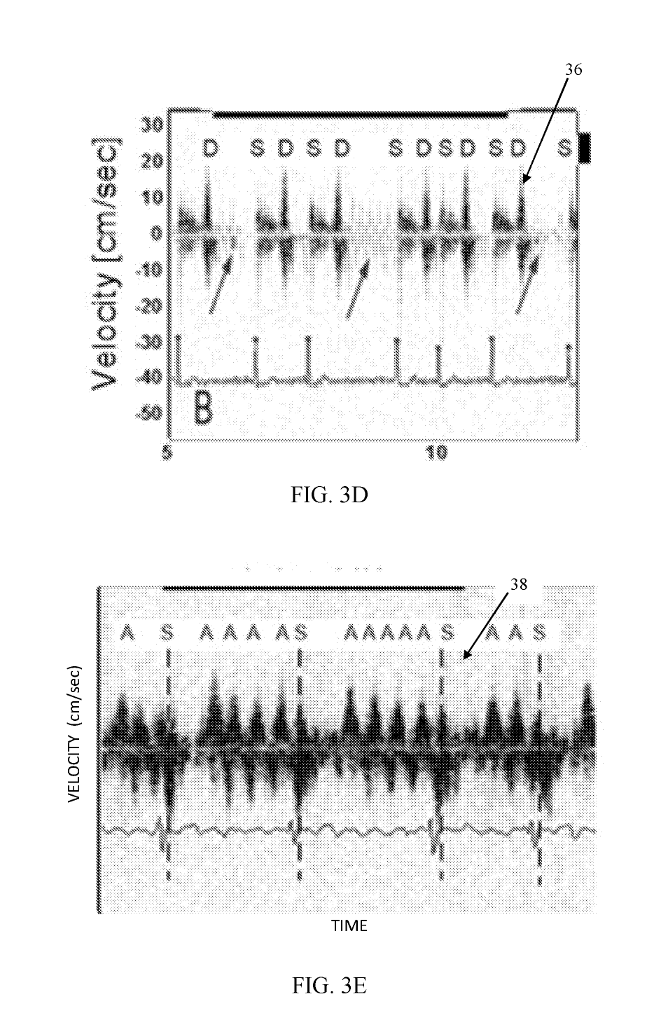

FIGS. 3A-3F are included to describe the theory of operation of the embodiments described herein. But it is important to note that the displays depicted in those figures are not actually generated by the D-Holter system that is worn by the patient. Instead, these figures depict the displays would be obtained if the LDS power and velocity data obtained by the D-Holter system were processed into a conventional Doppler ultrasound display in which the x axis represents time, the y axis represents velocity, and power is represented by color. (Note that in the figures, the conventional color display is replaced by grayscale for purposes of filing in this patent application.) Five different scenarios are depicted in FIGS. 3A-3F: normal heartbeats (FIG. 3A); heartbeats with an atrial extra systole (FIG. 3B); heartbeats with a ventricular extra systole (FIG. 3C); heartbeats with atrial fibrillation (FIG. 3D); and heartbeats with atrial flutter (FIG. 3E).

It has been postulated that the LDS represent movements generated by the cardiac mechanical activity that propagate through the lung along its vascular system. The Doppler system measures the movement velocity by the frequency shifts as well as the changes in the reflected ultrasound power amplitude. These reflected ultrasound waves, as picked up by the D-Holter system over the lung, are in the order of 80-100 dB, i.e. much stronger than the flow signals picked up by the standard Doppler systems from flow in blood vessels. This fact makes it possible to use the described simple patch transducers that rely on a single piezoelectric element, without the need for incorporating any focusing technology (e.g., by using a phased array transducer) into the system.

FIG. 3A shows that the LDS 30 for a normal heartbeat includes at least three distinct elements labeled S, D and A. These elements represent the mechanical movements associated with cardiac systole, diastole, and atrial contraction respectively. FIG. 3A also includes a conventional ECG trace (near the bottom) to illustrate the correlation between the various features (i.e., S, D, and A) of the LDS and the various features (e.g., an R wave) of the ECG. But it is important to note that the ECG traces that appear in FIG. 3A (and in the other figures in this application) are not actually generated by the embodiments described herein, and are included for reference and/or comparison purposes only to explain the theory of operation.

FIG. 3B shows that the LDS 32 for heartbeats with an atrial extra systole of sinus origin are registered in the D-Holter recordings and how they can be clearly identified by their distinct structure. More specifically, an additional full three element signal 32 (A+D+S) appears at some point during a normal cycle, interrupting the normal cycle.

FIG. 3C shows that the LDS 34 for heartbeats with a ventricular extra systole are registered in the D-Holter recordings. More specifically, an odd shaped long duration single element 34 interposes the normal sequence of events.

FIG. 3D depicts LDS tracings 36 recorded from a patient with Atrial Fibrillation (AF). This recording shows clear S and D signals. But the presystolic signal (labeled A in the normal tracing seen in FIG. 3A) is missing when AF occurs, as seen in FIG. 3D. The presence of this pattern 36 (i.e., the missing “A” signal) in the LDS recording makes it possible to detection AF by analyzing the LDS, and an algorithm for detecting this situation is described below.

FIG. 3E depicts LDS tracings 38 recorded and from a patient with Atrial Flutter (AFT). This recording shows a large number of extra “A” artifacts 38. The presence of this pattern in the LDS recording makes it possible to detection AFT by analyzing the LDS

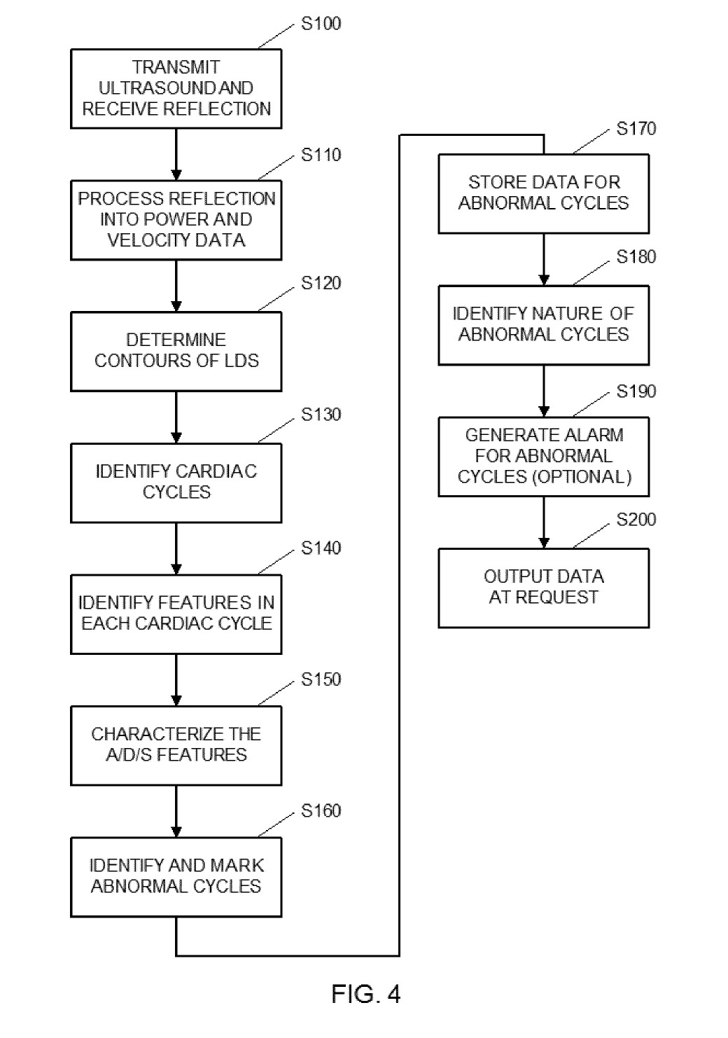

FIG. 4 is a schematic representation of the basic data handling procedure that is implemented by the processor 15 (shown in FIGS. 2A and 2B), and details of the various steps depicted in FIG. 4 are described below.

In step S100, ultrasound energy is transmitted into the patient, and the reflected ultrasound energy is received, in a conventional manner. In step S110, Doppler shifts in the received reflections are detected and processed into power and velocity data in a conventional manner, similar to the processing for conventional Doppler Sonograms. Note that because the Doppler returns from different positions on the patient's chest are similar, the placement of the transducer in an exact spot on the patient's chest in not necessary.

Conventional Doppler systems collect power and velocity data from many different depths or gates (e.g., 16 gates). But because the returns from different depths within the patient's lungs are roughly similar, D-Holter systems do not have to collect the Doppler data from multiple gates. Instead, the data from a single gate can be used for all subsequent processing described herein. This results in a significant decrease in the amount of data that must be processed. Optionally, the optimal gate or gates can be determined by analyzing the sonograms obtained from a few depths. Subsequently to this determination only the selected gate data will be stored.

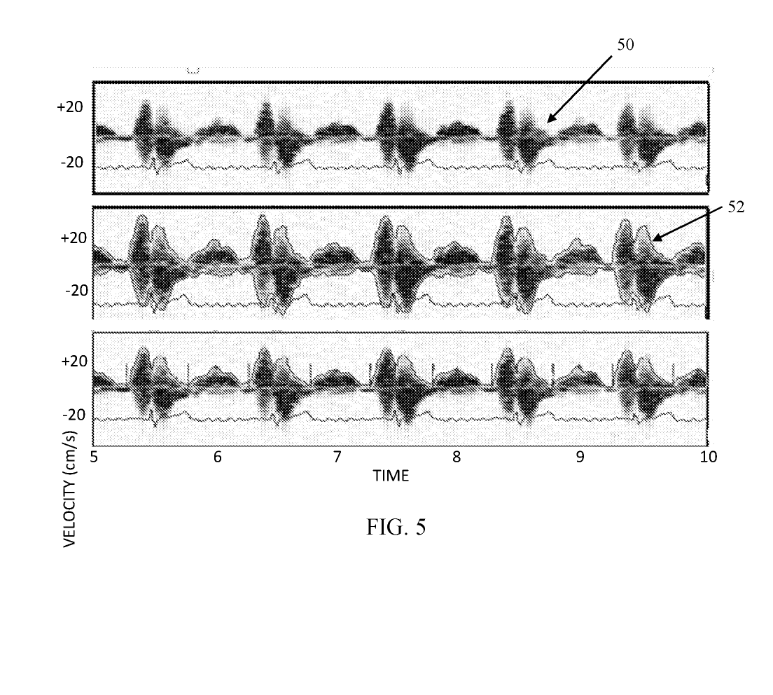

In step S120, the contours (i.e., envelope) of the LDS power and velocity data is determined using any conventional envelope-detecting algorithm. The top panel of FIG. 5 is an example of LDS power and velocity data 50 for a series of four heartbeats. And the trace 52 in the middle panel of FIG. 5 shows the contour (i.e., the envelope) of that LDS data. (Note once again that displays depicted in FIG. 5 are not generated by the D-Holter system. But they are included to explain what is happening in the various processing steps.)

In step S130, the cardiac cycles are identified. An assumption is made that when the D-Holter is connected to the patient and activated, the heart rate is usually operating in steady state and the LDS are usually stable and repetitive. If this is not the case (e.g., when an arrhythmia is actively occurring), a regular ECG would suffice to make the diagnosis. The benefits of D-Holter are larger when the arrhythmias are intermittent, especially when those arrhythmias occur at a very low frequency of incidence.

An adaptive approach is preferably used in order to keep up with any temporal changes during the monitoring time, such as when the heart rate (HR) increases (e.g., during exertion) or decreases (when the exertion ends). The step of identifying cardiac cycles is therefore preferably updated periodically (e.g. every 30-60 seconds) and the HR is re-estimated.

The identification of cardiac cycles without relying on an ECG signal is preferably based on estimating the heart rate (HR) using a Matched Filtering (MF) technique that involves one or more templates of LDS data that correspond to a normal cardiac cycle.

In some preferred embodiments that rely on MF, a pair of templates is used, with one template of the pair being used for slower HRs, and the other template of the pair being used for faster HRs. It is advantageous to use different templates for fast and slow HRs, because the expected features of normal LDS varies as a function of the HR. More specifically, as the heart beats faster, the “A” and “D” features in the LDS (as best seen in FIG. 3A) move towards each other and eventually merge together into what appears to be a single “A” feature.

In these preferred embodiments, the step of identifying the cardiac cycles (i.e., S130) includes two major stages: estimating the HR and match filtering. HR estimation may be implemented, for example, by autocorrelation of the contour of the spectrogram or the raw data. The peaks of the autocorrelation are detected and the average time difference between the peaks is calculated. The reciprocal of the average time is the estimated HR. The variance of the time difference between the peaks is also defined as the HR estimated variability. Once the HR is determined, a template for match filtering is selected based on whether the HR is greater than a threshold rate. A preferred threshold is an HR of 100, in which case one MF template would be selected when the HR is greater than 100 and the other MF template would be selected when the HR is less than 100. The envelope of the LDS is then match-filtered against the selected template. The purpose of this step is detecting the repeatability of a specific selected template. The output of the matched filtering is a continuous signal (or a digital representation thereof), the peak of which represents the start of each cardiac cycle.

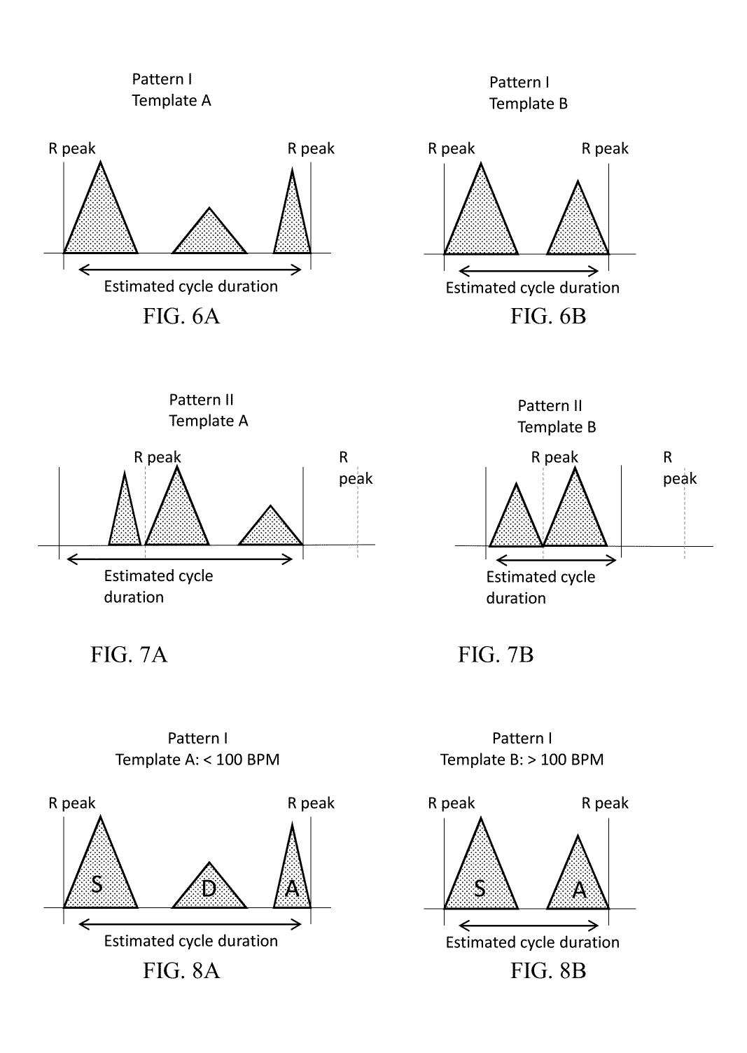

The calculation is conducted in either one of the following two cases: More specifically, when the HR is lower than the threshold, template A is used as the MF kernel, otherwise template B is used. In one preferred embodiment (referred to herein as the Pattern I embodiment), the templates in the pair have the shapes depicted in FIGS. 6A and 6B. In an alternative preferred embodiment (referred to herein as the Pattern II embodiment), the templates in the pair have the shapes depicted in the FIGS. 7A and 7B.

In either scenario, the template is flipped and convoluted with the LDS spectrogram contour or the LDS raw data to calculate the matched filter signal. The peaks of this signal are determined. A single cardiac cycle (i) is represented by a time frame that extends from [detected peak (i) time] and ends in [detected peak (i)+estimated cardiac cycle duration (1/HR)] time.

Alternative approaches for identifying the cardiac cycles may also be used. For example, the contour data that was determined in step 120 may be analyzed to determine the highest velocity that appears in the contour over a given time (e.g., 2 seconds), and the time at which that highest velocity was measured is deemed to be the start of a cardiac cycle. Because the LDS repeats in a periodic manner the vast majority of the time, the next point in time at which that same velocity appears (with a small tolerance of e.g., 5%) is deemed to be the start of the next cardiac cycle.

After identification of the cardiac cycles in step S130, processing proceeds to step S140, which is an optional step. In step S140, the various features of each cardiac cycle are identified. In the embodiment that uses Pattern I, the features are identified in two different ways, depending on the HR. More specifically, when the HR is lower than the HR threshold (discussed above); the “S” signal is defined as the signal in the first third of the cardiac cycle, the “D” signal is defined as the signal in the second third of the cardiac cycle, and the “A” signal is defined as the signal in the last third of the cardiac cycle. When the HR is more than the HR threshold; the “S” signal is defined as the signal in the first half of the cardiac cycle, the “A” is defined as the signal in the second half of the cardiac cycle, and the “D” signal is defined as Null. FIGS. 8A and 8B depict these definitions for the Pattern I embodiment.

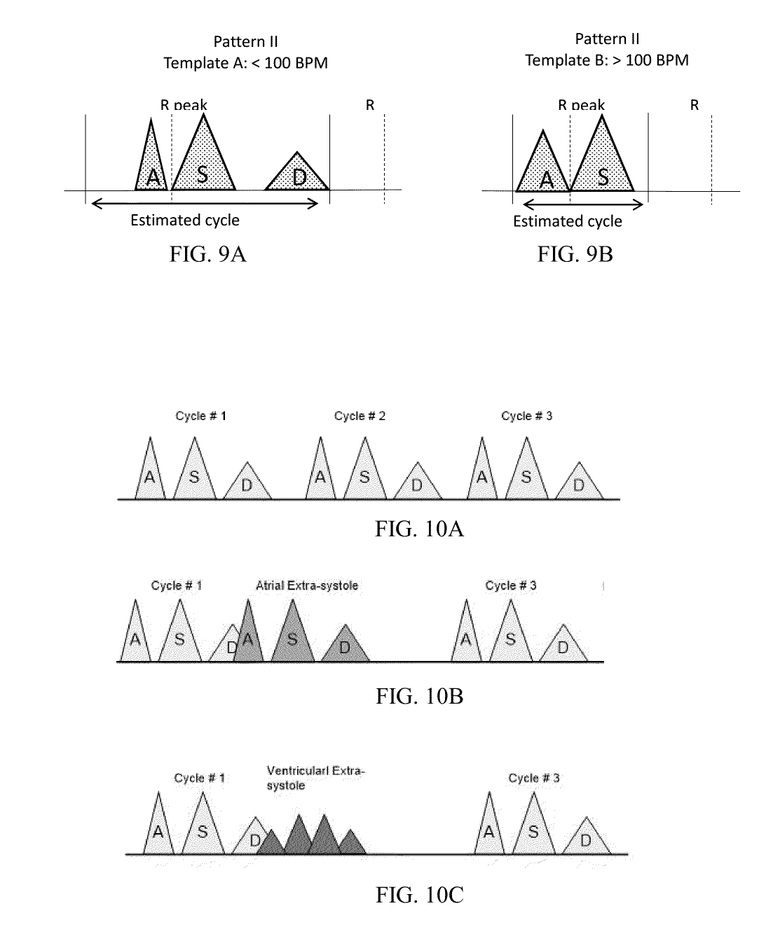

In the alternative embodiment that uses Pattern II, the features are also identified in two different ways, depending on the HR. When the HR is lower than the HR threshold; the “A” signal is defined as the signal in the first third of the cardiac cycle, the “S” signal is defined as the signal in the second third of the cardiac cycle, and the “D” signal is defined as the signal in the last third of the cardiac cycle. When the HR is more than the HR threshold; the “A” signal is defined as the signal in the first half of the cardiac cycle, the “S” is defined as the signal in the second half of the cardiac cycle, and the “D” signal is defined as Null. FIGS. 9A and 9B depict these definitions for the Pattern II embodiment.

After identification of the cardiac cycles in step S140, processing proceeds to step S150, which is also an optional step. In step S150, characterizations of the A, D, and S features (which were identified in step S140) in are calculated from the LDS. Examples of these characterizations include power integrals, durations, average velocities, peak velocities, slopes, etc.

In step S160, any cycle that is abnormal is identified and marked. One example of an algorithm that may be used to determine which cycles are abnormal is to define normal cycles as one of the patterns used above (template A or template B), depending on the HR. All other patterns are defined as “Abnormal” cycles. Optionally, a support-vector-machine (SVM) based classifier may be used to implement this step. In this situation, the SVM is preferably trained offline to differentiate between the two classes; Normal and Abnormal cycles, using its features. The product of the learning (training) stage is a mathematical model which is used online to differentiate (classify) between these classes, preferably using a matched filter.

In alternative embodiments, the decision to classify a cycle as abnormal may be based on a set of rules. Examples of rules that may be used to classify a cycle as abnormal include: (a) cycles in which the measured HR differs from an adaptive estimation of HR that is based on the HR of the previous few cycles by an amount that is larger than a threshold (e.g. 20%); (b) If the adaptive HR estimation switches from using pattern A to B, or vice versa; (c) If the estimated HR exceeds an upper threshold (e.g. 120 BPM) or falls below a lower threshold (e.g., 40 BPM); (d) if the features identified in step S140 do not match an expected set of features for a given HR (e.g., if an expected feature is missing, or if an unexpected extra feature is present; or (e) if a characterization of a feature calculated in step S150 has an unexpected value (e.g., if the duration of a feature exceeds an expected value by a threshold percentage). Cycles that do not meet one of the rules for an “abnormal” cycle are classified as normal.

In step S170, data for any cycle that has been identified in step S160 as being abnormal is stored in the memory 16 (shown in FIGS. 2A and 2B). A time stamp that identifies the time of the abnormal cycle is preferably stored together with the data for the abnormal cycle. In some embodiments, only the power and velocity data for the abnormal cycle is stored. In these embodiments, there is no need to determine the nature of the abnormality in real time in the D-Holter device that is being worn by the patient. Instead, the nature of the abnormality can be determined by an external device at a later time. This may be accomplished at the end of the testing period, for example, by outputting the power and velocity data and associated time stamps for all abnormal cycles to the external device, so that the external device can analyze the data (and/or display the data so that a human operator can determine the nature of the abnormality).

In those embodiment that perform the steps of identifying features in the cardiac cycle (step S140, discussed above), the storing step S170 preferably includes storing data for each abnormal cycle indicating which features were identified in step S140. In those embodiment that perform the steps of characterizing features in the cardiac cycle (step S150, discussed above), the storing step S170 preferably includes storing the characterizations for the features were characterized in step S150. In these embodiments, the power and velocity data for the abnormal cycle may also be stored in memory.

Notably, there is no need to store any data for any of the normal cycles. This dramatically reduces the memory that must be include in system, because the vast majority of cycles will be normal cycles. This is especially important when the power and velocity data itself is stored in memory, because that data is relatively large.

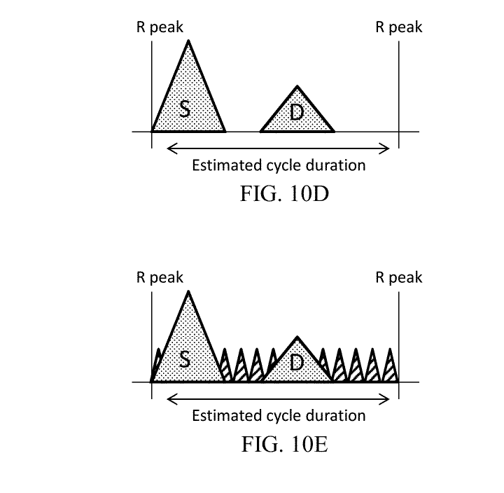

In step S180, which is an optional step, the nature of the abnormal cycle is identified. Examples of abnormal cycles include atrial extra systoles, ventricular extra systoles, atrial fibrillation (AF), and atrial flutter (AFT), and expected feature patterns for normal heartbeats and the four abnormal patterns mentioned above are shown in FIGS. 10A-10E, respectively. For example, as compared to the expected normal set of features which is shown in FIG. 10A, the “A” feature is missing at the end of the cardiac cycle in AF (FIG. 10D), and a large number of extra “A” features are present in AFT (FIG. 10E). Optionally, within the set of “abnormal” cycles classified previously, the SVM may be used with a different models to identify which of the various abnormalities or arrhythmias is present. Any deviation from the normal expected patterns is recognized.

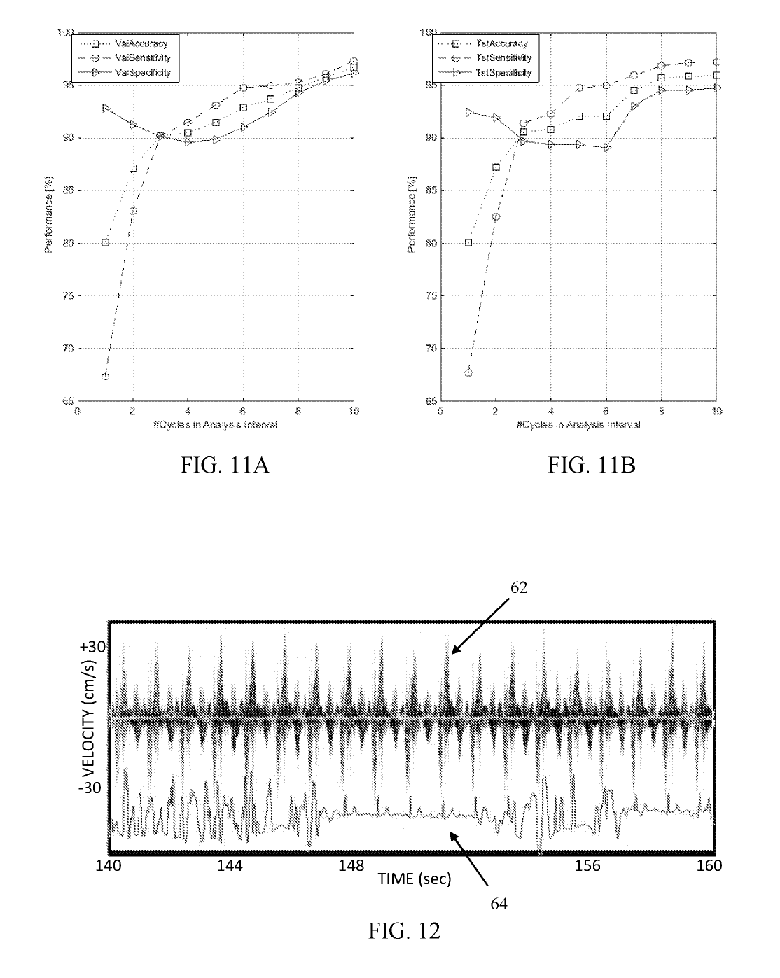

FIGS. 11A and 11B represent one example of performance measures obtained by an SVM classifier for recognizing AF. Sensitivity, Specificity and Accuracy are used as performance measures. More specifically, FIG. 11A represents the performance obtained while learning and training using a validation set (using a set that included ⅔ of a set of 325 cardiac cycles known to represent AF, and 325 cardiac cycles of non-AF). Assuming that the SVM is trained properly, the validation performance will be a good estimate for the future performance of the SVM on unseen sets of new data.

FIG. 11B represents the performance obtained while using the SVM with the pre-trained model from the validation set on the remaining ⅓ of the set of 325 cardiac cycles known to represent AF, plus the 325 cardiac cycles of non-AF. Both plots (FIGS. 11A and 11B) show similar behavior, indicating that the learnt model is general enough to correctly classify previously unseen new data.

The testing depicted in FIGS. 11A and 11B was achieved as follows: The sonograms of five AF subjects and eight non-AF subjects were recorded and sampled at 3 kHz, for a duration of 325 cardiac cycles for each subject. An algorithm that calculates the power integral in 80 msec windows that precede the start of S feature was activated on the data. SVM was used classify AF vs. non-AF cycles. As seen in FIG. 11, three consecutive cycles are identified with 90% accuracy/sensitivity/specificity within the string of normal cycles. These results establish that D-Holter can advantageously diagnose AF with a very high degree of certainty, even when the fibrillation episode is extremely short (e.g., only 2-4 cycles embedded in a large number of normal cycles).

Similar performance can be expected in patients with atrial flutter (AFT). In these cases (see FIG. 10D), the excitatory electric signals are regular but very rapid such that the atria contract in synchrony but at a very high pace (as high as 400 contractions/min). Under these conditions the cardiac conducting system cannot cope with the high rate and responds in ventricular contraction of much lower pace. Note that in this case, the electric activity that would be reflected in an ECG would be hard to diagnose in noisy recordings and short episodes. In contrast, the LDS recordings for patients with AFT show a chain or multiple pronounced signals (labeled A in FIG. 10D) that dominate the tracing. The flutter signals that represent the synchronous atrial contraction are very distinct and easily recognizable. The D-Holter system is therefore superior to conventional ECG-based Holter systems for diagnosing AFT as well.

Returning now to FIG. 4, processing continues is step S190, which is also an optional step. In step S190, an alarm or another indicator is used to notify the patient or medical personnel that an abnormal cycle has been detected. The alarm may include audible and/or visual alerts. Optionally, after a predetermined number of abnormal cycles (e.g., 5-10) have been detected, the patient may be notified that enough data has been collected, and the data collection process can be ended early. The notification may be accomplished using include audible and/or visual alerts. This will allow the patient to avoid wearing the D-Holter device longer than necessary, to minimize discomfort to the patient and cost.

After enough data has been collected (e.g., after 48 hours have elapsed) or after the predetermined number of abnormal cycles are detected, data collection stops, and the collected data is output in step S200. Returning to FIGS. 2A and 2B, this may be accomplished by having the processor 15 read the data that was stored in memory in step S170 to an external or remote computer via any conventional interface, such as a wired interface that uses connector 10 and/or a wireless interfaces (not shown).

An important advantage of D-Holter relates to detecting the conditions of AF and AFT. AF is a highly prevalent condition in people above 65. It is the result of desynchronized electric activity and as a result desynchronized contraction of different areas in the atria. The uncoordinated contractions render the atrial contraction ineffective and thus reduce the cardiac performance. Furthermore, AF may result in the formation and dissemination of blood thrombi that may pose a serious medical problem such as pulmonary embolism.

The normal electric activity associated with atrial contraction, the P wave of the ECG, is small and sometimes hard to detect. In AF a minute irregular oscillation replaces the P wave. This abnormal electric activity is often very difficult to detect, especially in noisy recordings, and when the AF is interrupted by long intervals between fibrillatory episodes. In such cases the conventional ECG based Holter recording time needs to be very long in order to be sufficient for detection. However, the conventional ECG based Holter wearing duration usually does not extended beyond 24-48 hours in view of the described inconvenience to the patient, in which case the AF condition may not be detected. This problem is overcome by using the D-Holter for two reasons: First, it is much more convenient to use as it requires only one electrode rather than the multi-electrode and complex wiring that are required by the conventional ECG based Holter monitors; and second, the AF condition is easier to detect based on the more obvious abnormality in the LDS (as opposed to the more subtle abnormality in the P wave of the ECG signals).

Another advantage of D-Holter over the conventional ECG based Holter systems is due to the fact that the D-Holter records the mechanical activity of the heart rather that the electric activity associated with the heart. The D-Holter signals therefore provided a clearer indication of each cardiac cycle, and its main components, from which cardiac rhythm, pulse intervals, etc. can be determined.

Another advantage of D-Holter over the conventional ECG based Holter is that LDS obtained from different positions on the chest wall have very similar characteristics. Therefore, in contrast to conventional ECG based Holter, relatively small transducer movements with respect to the chest will not result in significant recording changes or movement artifacts in D-Holter systems.

Yet another advantage of D-Holter over the conventional ECG based Holter is that D-Holter measurements are much less sensitive to noise generated by electric equipment and by EMG generated by the chest muscles. FIG. 12 provides an example of how the readings obtained from both an LDS-based system and a conventional ECG-based system can change in the presence of patient movement. Note how the LDS (the upper trace 62) remains relatively constant even though the patient is moving, while the ECG (the lower trace 64) drops out between t=147 and t=153 when the patient is moving.

Note that the embodiments described above are used to diagnose various cardiac abnormalities without relying on conventional ECG measurements. However, in alternative embodiments, the processing of the LDS described above may be combined with a conventional ECG-based system to obtain two different modalities of information simultaneously. Such embodiments may be useful to detect mechano-electric dissociation.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

I claim:

1. An apparatus for monitoring the operation of a heart of a patient, the apparatus comprising:
an ultrasound transducer configured to transmit ultrasound energy into the lungs of the patient and receiving ultrasound energy reflected from the lungs of the patient;
an ultrasound processor configured to detect Doppler shifts in the received reflections and process the Doppler shifts into power and velocity data;
a memory configured to store data; and
a processor configured to identify cardiac cycles based on the power and velocity data, determine when an identified cardiac cycle is abnormal, store data corresponding to the abnormal cardiac cycle in the memory when a cardiac cycle is abnormal, and output the stored data.

2. The apparatus of claim 1, wherein the processing of Doppler shifts into power and velocity data is implemented using an algorithm designed to increase signal from moving borders between blood vessels in the lung and air filled alveoli that surround the blood vessels, with respect to other reflected ultrasound signals.

3. The apparatus of claim 1, wherein the processor is further configured to identify features in a plurality of cardiac cycles, wherein the features in any given cardiac cycle are identified after the given cardiac cycle has been identified.

4. The apparatus of claim 1 wherein the processor is further configured to identify a nature of the abnormality after making the determination that a cardiac cycle is abnormal.

5. The apparatus of claim 1, wherein the processor is further configured to identify cardiac cycles by determining an envelope of the power and velocity data and identify cardiac cycles based on the determined envelope.

6. The apparatus of claim 1, wherein the processor is further configured to determine when an identified cardiac cycle is abnormal by match filtering using a match filter kernel that corresponds to a normal heartbeat.

7. The apparatus of claim 6, wherein the match filter kernel includes a first feature that corresponds to systole, a second feature that corresponds to diastole, and a third feature that corresponds to atrial contraction.

8. The apparatus of claim 1, wherein the processor is further configured to determine when an identified cardiac cycle is abnormal by match filtering using a first match filter kernel when the patient's heartrate is below a threshold rate, and match filtering using a second match filter kernel when the patient's heartrate is above the threshold rate.

9. The apparatus of claim 8, wherein the first match filter kernel includes a first feature that corresponds to systole, a second feature that corresponds to diastole, and a third feature that corresponds to atrial contraction, and wherein the second match filter kernel includes a first feature that corresponds to systole and a second feature that corresponds to diastole but does not include a feature that corresponds to atrial contraction.

10. The apparatus of claim 1, wherein the processor is further configured to determine when an identified cardiac cycle is abnormal by determining when the identified cardiac cycle includes at least one of atrial fibrillation and atrial flutter.

11. A method of monitoring the operation of a heart of a patient, the method comprising the steps of:
transmitting ultrasound energy into the lungs of the patient;
receiving ultrasound energy reflected from the lungs of the patient and detecting Doppler shifts in the received reflections;
processing the Doppler shifts into power and velocity data;
identifying cardiac cycles based on the power and velocity data;
determining when an identified cardiac cycle is abnormal;
storing, when a determination is made that a cardiac cycle is abnormal, data corresponding to the abnormal cardiac cycle; and
outputting the data that was stored in the storing step.

12. The method of claim 11, wherein the step of processing the Doppler shifts into power and velocity data includes an algorithm designed to increase signal from moving borders between blood vessels in the lung and air filled alveoli that surround the blood vessels, with respect to other reflected ultrasound signals.

13. The method of claim 11, further comprising the step of identifying features in a plurality of cardiac cycles, wherein the features in any given cardiac cycle are identified after the given cardiac cycle has been identified.

14. The method of claim 11, further comprising the step of identifying, after a determination is made that a cardiac cycle is abnormal, a nature of the abnormality.

15. The method of claim 11, wherein the step of identifying cardiac cycles comprises the steps of:
determining an envelope of the power and velocity data; and
identifying cardiac cycles based on the determined envelope.

16. The method of claim 11, wherein the step of determining when an identified cardiac cycle is abnormal comprises the step of match filtering using a match filter kernel that corresponds to a normal heartbeat.

17. The method of claim 16, wherein the match filter kernel includes a first feature that corresponds to systole, a second feature that corresponds to diastole, and a third feature that corresponds to atrial contraction.

18. The method of claim 11, wherein the step of determining when an identified cardiac cycle is abnormal comprises the steps of:
match filtering using a first match filter kernel when the patient's heartrate is below a threshold rate; and
match filtering using a second match filter kernel when the patient's heartrate is above the threshold rate.

19. The method of claim 18, wherein the first match filter kernel includes a first feature that corresponds to systole, a second feature that corresponds to diastole, and a third feature that corresponds to atrial contraction, and wherein the second match filter kernel includes a first feature that corresponds to systole and a second feature that corresponds to diastole but does not include a feature that corresponds to atrial contraction.

20. The method of claim 11, wherein the step of determining when an identified cardiac cycle is abnormal comprises the step of determining when the identified cardiac cycle includes at least one of atrial fibrillation and atrial flutter.
